(19) United States
(12) Patent Application Publication
(10) Pub. No.: US 2024/0359029 A1
(43) Pub. Date: Oct. 31, 2024

(54) PHOTOTHERAPY DEVICE FOR TREATING VITAMIN D DEFICIENCY

(71) Applicant: LUMITEX, INC, Strongsville, OH (US)

(72) Inventors: Michael KERNS, Strongsville, OH (US); Alan GRESZLER, Strongsville, OH (US); Mark REA, Melrose, NY (US); Andrew BIERMAN, Albany, NY (US)

(73) Assignee: LUMITEX, INC, Strongsville, OH (US)

(21) Appl. No.: 18/686,279

(22) PCT Filed: Aug. 30, 2022
(86) PCT No.: PCT/US2022/075626

§ 371 Date: Feb. 23, 2024

Related U.S. Application Data
(60) Provisional application No. 63/239,636, filed on Sep. 1, 2021.

(51) Int. Cl. A61N 5/06; A43B 3/36

(52) U.S. Cl. CPC A61N 5/0616; A43B 3/36; A61N 2005/063; A61N 2005/0645; A61N 2005/0652; A61N 2005/0661; A61N 2005/0663

(57) ABSTRACT

A phototherapeutic footwear is provided for treating vitamin D deficiency using UVB light and red light. The phototherapy device includes a light source configured to emit UVB light to stimulate production of vitamin D by a patient's body and to emit red light to stimulate melatonin production by the patient's body. The light source and a power source are mechanically supported by a main body, such that the UVB light and red light are directed onto a skin of a patient.

### RELATED APPLICATIONS

This application claims the benefit of U.S. 63/239,636 filed on Sep. 1, 2022. Which is herein incorporated by reference in its entirety.

### TECHNICAL FIELD

The present disclosure relates generally to treating vitamin D deficiency and more particularly to a phototherapy device for treating vitamin D deficiency.

### BACKGROUND

Vitamin D is used by the body in association with calcium (e.g., in bone formation). Most popularly, vitamin D deficiency is associated with rickets, a disease in which the bone tissue doesn't properly mineralize, leading to soft bones and skeletal deformities. The importance of vitamin D is becoming increasingly understood, with research pointing to the importance of vitamin D in protecting against a host of health problems.

When your skin is exposed to sunlight, it manufactures vitamin D. The sun's ultraviolet B (UVB) rays interact with a protein called 7-DHC in the skin, converting it into vitamin D3, the active form of vitamin D.

### SUMMARY

The present disclosure provides a phototherapeutic footwear for treating vitamin D deficiency using a light source emitting UVB light to stimulate production of vitamin D by a patient's body and emitting red light to stimulate melatonin production by the patient's body.

While a number of features are described herein with respect to embodiments of the invention; features described with respect to a given embodiment also may be employed in connection with other embodiments. The following description and the annexed drawings set forth certain illustrative embodiments of the invention. These embodiments are indicative, however, of but a few of the various ways in which the principles of the invention may be employed. Other objects, advantages and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention in which similar reference numerals are used to indicate the same or similar parts in the various views.

FIG. 1 is a side perspective view of an exemplary embodiment of a phototherapeutic footwear having multiple light emitters.

FIG. 2 is a bottom perspective view of the phototherapeutic footwear of FIG. 1.

FIG. 3 is a side cut away view of the phototherapeutic footwear of FIGS. 1 and 2.

FIG. 4 is a front view of an exemplary embodiment of a phototherapeutic footwear including a light source and light guides.

FIG. 5 is a rear view of the phototherapeutic footwear of FIG. 4.

FIG. 6 is a side view of an exemplary embodiment of a phototherapeutic footwear comprising a slipper or shoe.

FIG. 7 is a cut away view of the phototherapeutic footwear of FIG. 6.

FIG. 8 is a perspective view of another exemplary embodiment of a phototherapeutic footwear.

The present invention is described below in detail with reference to the drawings. In the drawings, each element with a reference number is similar to other elements with the same reference number independent of any letter designation following the reference number. In the text, a reference number with a specific letter designation following the reference number refers to the specific element with the number and letter designation and a reference number without a specific letter designation refers to all elements with the same reference number independent of any letter designation following the reference number in the drawings.

### DETAILED DESCRIPTION

According to a general embodiment, a phototherapeutic footwear is provided for treating vitamin D deficiency using UVB light and red light. The phototherapy device includes a light source configured to emit UVB light to stimulate production of vitamin D by a patient's body and to emit red light to stimulate melatonin production by the patient's body. by co-activating the production of vitamin D and melatonin, it is possible to obtain synergistic effects due to melatonin's role in enhancing the signaling of vitamin D. The light source and a power source are mechanically supported by a main body, such that the UVB light and red light are directed onto a skin of a patient.

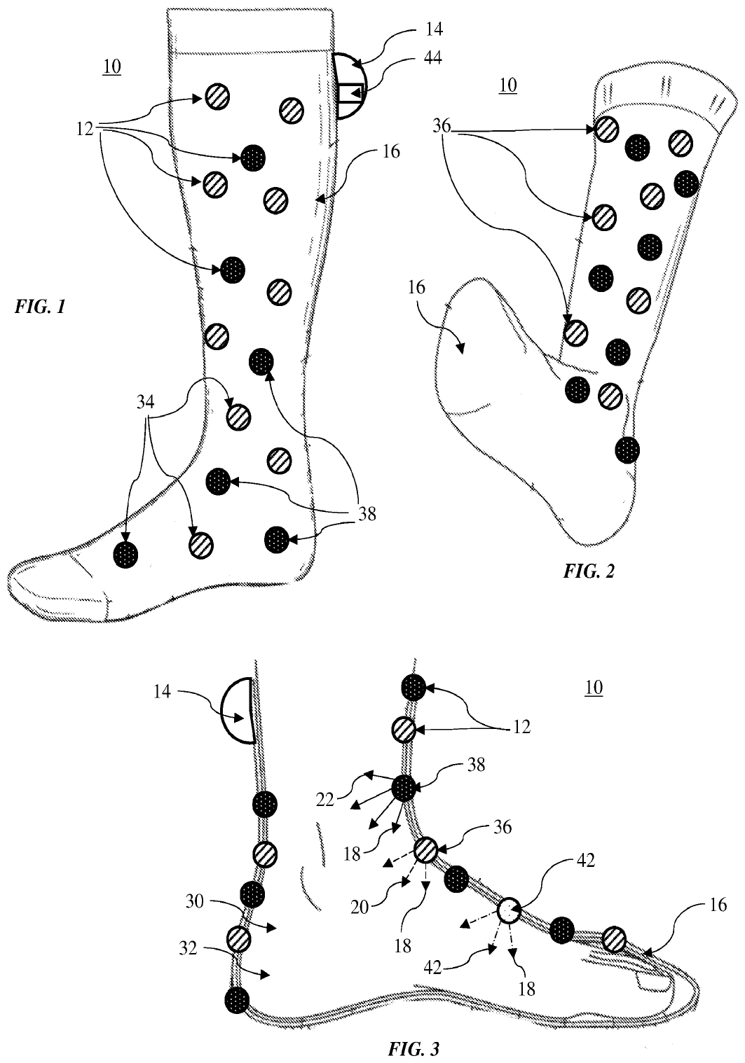

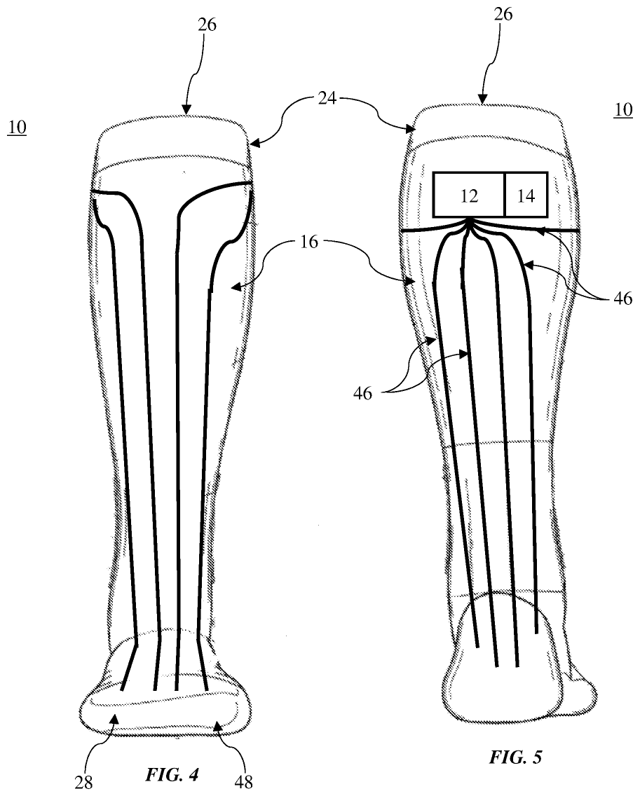

An exemplary phototherapeutic footwear 10 for treating vitamin D deficiency is shown in FIGS. 1, 2, and 3. The phototherapeutic footwear 10 includes a light source 12, a power source 14, and a main body 16. The light source 12 emits phototherapeutic light 18 including UVB light 20 for stimulating vitamin D production and red light 22 for stimulating melatonin production. The UVB light 20 includes light having a wavelength within a range of 300-315 nm and the red light 20 includes light having a wavelength within a range of 620-700 nm. The power source 14 stores and supplies electrical power to the light source 12. The main body 16 mechanically supports the light source 12 and the power source 14.

The main body 16 includes a proximal end 24 having an opening 26, a distal end 28, and an interior 30 bounded by the main body 16. The main body 16 is configured to receive a foot 32 and/or leg of a patient. The main body 16 supports the light source 12 such that the phototherapeutic light 18 is emitted into the interior 30 of the main body 16.

As shown in FIGS. 1-3, the light source 12 may include a plurality of light emitters 34. The light emitters 34 may include UVB light emitters 36 and red light emitters 38. The UVB light emitters 36 emit the UVB light 20 and the red light emitters 38 emit the red light 22. The light emitters 34 may be spread across the main body 16 such that the UVB light 20 and the red light 22 are received across different areas of the foot 32 when inserted into the interior 30. For example, the light emitters 34 may be spread such that the phototherapeutic light 18 is received by at least two of a top of the foot, a heel of the foot, a calf of the patient, and a shin of the patient. The phototherapeutic light may be uniformly delivered over a defined surface area. The light emitters 34 are electrically connected to (e.g., via wires embedded in a material of the main body 16) and receive electrical power for emitting the phototherapeutic light 18 from the power source 14.

The light emitters 34 may also include blue light emitters 40 that emit germicidal blue light 42. A wavelength range of the blue light 42 includes a wavelength of 405 nm. For example, the blue light 42 may include the wavelength 405 nm and additional wavelengths.

As shown in FIGS. 4 and 5, the phototherapeutic footwear 10 may include light guides 46 extending from the light source 16 towards at least one of the proximal end 24 or the distal end 28. The light guides 16 are positioned to receive the emitted phototherapeutic light 18 from the light source 16. The light guides 16 transmit the phototherapeutic light via total internal reflection and emit the phototherapeutic light 18 into the interior 30 of the main body 16. For example, the power source 14 and the light source 12 may be located nearer the opening 26 than a closed toe portion 48 and the light guides 46 may extend) from the light source 16 towards the closed toe portion 48. The light guides 46 may extend at least one of longitudinally or circumferentially along the main body 16.

In one embodiment, the light source includes UVB light emitters 36 and red light emitters 38. The light emitters 34 may be positioned to emit the phototherapeutic light 18 such that the phototherapeutic light 18 is received by a light receiving end of the light guides 16. Each of the light guides 46 may receive phototherapeutic light 18 from a single type of light emitter (e.g., one or more UVB light emitters 36 or one or more red light emitters 38). Alternatively, at least a portion of the light guides 46 may receive phototherapeutic light 18 from more than one type of light emitter.

The main body 16 may be at least partially made of a woven fabric and the light guides 46 may be interwoven into the woven fabric. For example, the light guides 46 may be included as a woven strand in the woven fabric. Alternatively, the light guides 46 may be attached to an interior surface of the main body 16. As an example, the main body 16 may be made of a dual layer of woven fabric and the light guides 46 may be sandwiched between the layers of the dual layer of woven fabric.

The phototherapeutic footwear 10 may additionally include processor circuitry 44 for controlling the emission of the phototherapeutic light 18. The processor circuitry 44 may control the UVB light emitters 36 separately from the red light emitters 38. For example, the processor circuitry 44 may independently operate the UVB light emitters 36 (i.e., modulate the emission of the UVB light 20) and the red light emitters 38 (i.e., modulate the emission of the red light 22). Similarly, the processor circuitry 44 may independently operate the blue light emitters 40 from the UVB light emitters 36 and the red light emitters 38.

In one embodiment, the processor circuitry 44 separately controls a dosage of the UVB light 20 emitted by the light source 16 and a dosage of the red light 22 emitted by the light source 16. For example, the processor circuitry 44 may measure a dosage of the UVB light 20 emitted by the light source 16. The processor circuitry 44 may also measure a dosage of the red light 22 emitted by the light source 16. The processor circuitry 44 may measure a dosage of a type of light (e.g., UVB light 20 or red light 22) based on an amount of time (i.e., a time duration) over which the type of light was emitted. For example, the processor circuitry 44 may keep track of a total time duration that UVB light 20 was emitted by the UVB light emitters 36 over a 24 hour period. The processor circuitry 44 may use this time duration to calculate a dose of UVB light 20 by multiplying the time duration by a known factor.

The processor circuitry 44 may also turn off a light emitter when the dosage of the associated light exceeds a threshold. For example, when the measured dosage of the UVB light 20 is greater than or equal to a UVB dosage threshold, the processor circuitry 44 may stop the emission of the UVB light 20 (e.g., for the remainder of the day). Alternatively, or additionally, when the measured dosage of the red light 22 is greater than or equal to a red dosage threshold, the processor circuitry 44 may stop the emission of the red light 22. For example, the processor circuitry 44 may limit dosage by specifying the UVB dosage threshold as a duration of time and limit the cumulative amount of time that the UVB light emitters 36 emit the UVB light 20 to less than the UVB dosage threshold. Similarly, comments apply concerning the red light emitters 38 and red light 22.

In one embodiment, the phototherapeutic footwear includes a dosage sensor (also referred to as a dosimeter) configured to sense a dosage of at least one of UVB light or red light received by the patient. For example, the dosage sensor may sense a dosage of UVB light and/or red light received from the ambient environment. This sensed dosage may be added to the dosage of the UVB light and/or red light and used to determine when the UVB dosage threshold and/or red dosage threshold has been met.

The processor circuitry 44 may also limit emission of a type of light based on a time of day. For example, the processor circuitry 44 may determine a time of day (e.g., using an internal or external clock) and compare the time of day to a red light initiation threshold. The red light initiation threshold may identify an earliest time for beginning emission of the red light 22 by the red light emitters 38. When the time of day is after the red light initiation threshold, the processor circuitry may cause the light source 12 to emit the red light 22. The red light initiation threshold may be set using any suitable method (e.g., based on typical environmental increase in proportion of red light during the day/night cycle).

The phototherapeutic footwear 10 may also include a sensor 50 for detecting when the foot 32 is inserted into the opening 26. The sensor 50 may be used to minimize the phototherapeutic light 18 being transmitted into the external environment (i.e., outside of the main body 16) by detecting when the opening 26 is obstructed and preventing the phototherapeutic light 18 from being transmitted into the external environment through the opening 26. The processor circuitry 44 may stop the emission of the UVB light 20 when the sensor 50 detects that the foot 32 is not inserted into the opening 26. For example, the sensor 50 may detect an obstruction in the opening 26. When the sensor 50 detects an obstruction, the processor circuitry 44 may enable the light source 16 to emit the phototherapeutic light 18. Conversely, when the sensor 50 detects that the opening 26 is not obstructed (i.e., a foot 32 or some other object is not inserted into the interior 30 of the main body 16), the processor circuitry 44 may disable the light source 16, such that the phototherapeutic light 18 is not emitted (e.g., the processor circuitry 44 may not send a signal to the light source 16 to emit the phototherapeutic light 18).

The sensor 50 may be any suitable device for detecting when a foot 32 is inserted into the opening 26. For example, the sensor 50 may be a photodetector (e.g., one or more photodetectors located at the proximal end 24 of the main body 16) for detecting light. When the phototherapeutic footwear 10 is worn by a patient, the patient's leg and/or foot may block the sensor 50, such that the sensor 50 does not detect light. Conversely, when the phototherapeutic footwear 10 is not being worn, the sensor 50 may be unobstructed such that light is detected by the sensor 50.

The processor circuitry 44 may initiate emission of the phototherapeutic light 18 when the sensor 50 detects at a current time point that the foot 32 is inserted (i.e., newly inserted) into the opening 26 after the sensor 50 detected at a previous time point that the foot 32 was not inserted into the opening 26. That is, the processor circuitry 44 may initiate emission of the phototherapeutic light 18 (e.g., at least one of the UVB light 20 or the red light 22) when the user puts the phototherapeutic footwear 10 on. The processor circuitry 44 may detect when a user puts the phototherapeutic footwear 10 on by the sensor 50 transitioning from indicating that a foot 32 is not detected (i.e., a foot is not inserted into the opening 26) to indicating that a foot 32 is detected.

The phototherapeutic footwear 10 may be any suitable footwear covering a portion of a patient's foot 32. In the embodiments shown in FIGS. 1-5, the main body 16 is a sock having a closed toe portion 48 at the distal end 28 opposite the opening 26. The power source 14 may be located nearer the opening 26 than the closed toe portion 48.

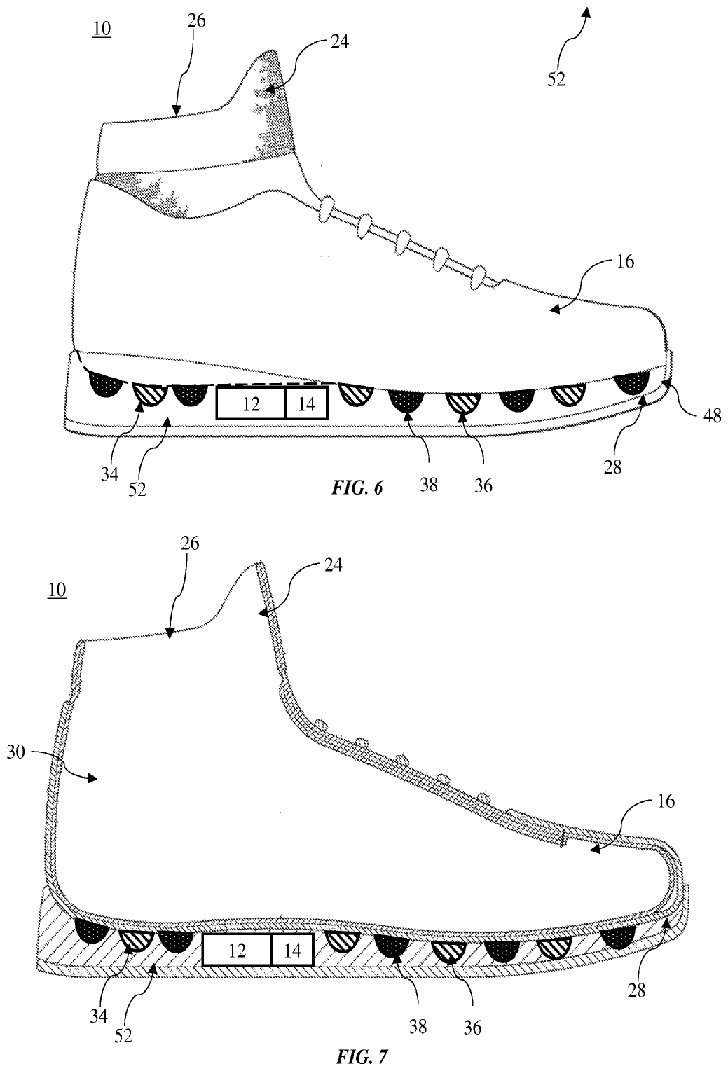

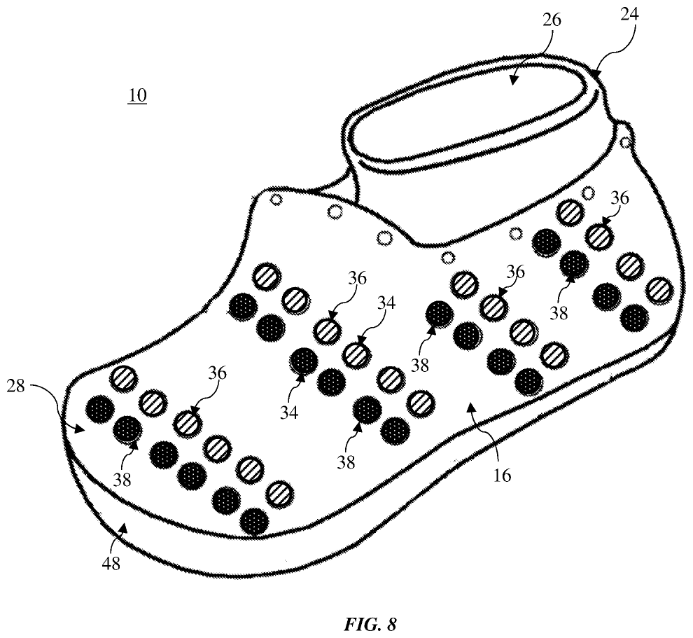

In another embodiment, as shown in FIGS. 6, 7, and 8, the main body 16 may be a slipper or shoe having a sole 52. The power source 14 may be housed within the sole 52. The processor circuitry 44 and/or light source 16 may also be housed with the sole. For example, the light emitters 34 may be located in a sole 52 of the main body 16 and illuminate a sole of the foot 32 of the patient.

The main body 16 may completely or partially enclose the interior 30 of the main body 16. For example, the main body 16 may have an open toe or may include other openings. Alternatively, as shown in FIGS. 1-8, the main body 16 may have a single opening 26 and a closed toe.

The light guides 46 may take the form of a flat fiber insert and the light source 16 may be arranged in a strip that is aligned along one edge of the insert. With a series of light emitters 34 (e.g., LEDs), the phototherapeutic light 18 may be well distributed, and a rudimentary style of coupling optic may be used (e.g., a molded acrylic strip with a series of indents on one side to receive the light emitters 34 and on the other side a cavity to receive the light receiving ends of the light guides 46).

The light guide 46 may be made of any suitable material having a structure, such that light emitted by the light source 16 and received by the light guide 46 is propagated via total internal reflection. For example, the light guide 46 may comprise a series of optical fibers (e.g., made from glass or plastic). The light guides 46 may include multiple separate structures (e.g., fiber optics) or a single structure (e.g., flat fiber).

The light emitters 34 may comprise light emitting diodes (LEDs), micro-LEDs, organic LEDs (OLEDs), polymer light emitting diodes (PLED)), a mercury lamp (e.g., including a spectral filter), or any suitable source of light. For example, a clad flat fiber and LEDs may be encased in a gel. Electrical connection may be made to the LEDs at an edge of the gel. In an alternative embodiment, micro-LEDs (without a separate light guide) may be embedded in the gel. Similarly, electrical connection to provide power to the micro-LEDs may be made at a surface of the main body 16.

The UVB light emitter 36 may be any suitable source of UVB light. In one embodiment, the UVB light has a spectrum of 310 nm+/−5 nm for the purpose of stimulating vitamin D through skin exposure. The UVB light spectrum may alternatively be 310 nm+/−10 nm or 310 nm+/−20 nm, etc. The spectrum may be chosen as representing an optimum ratio of vitamin D therapeutic benefit vs erythema risk.

The red light emitter 38 may be any suitable source of red light. In one embodiment, the UVB light has a spectrum of 670 nm+/−5 nm for the purpose of stimulating melatonin production through skin exposure. The red light spectrum may alternatively be 660 nm+/−10 nm or 660 nm+/−20 nm, etc.

The power source 14 may be any suitable source of electrical power. For example, the power source 14 may be a battery mechanically supported by the main body 16. The power source 14 may be releasably attached to a clip attached to the main body 16. Alternatively or additionally, the power source 14 may be stored in a pocket of the main body 16.

The processor circuitry 44 may modulate the amount of phototherapeutic light 18 delivered to the skin via the light source 16. For example, the processor circuitry 44 may control at least one of a duration, pattern, wavelength, or intensity of phototherapeutic light 18 emitted by the light source 16. The processor circuitry 44 may modulate the amount of phototherapeutic light 18 to provide a therapeutically effective dose to treat vitamin D deficiency.

The processor circuitry 44 may have various implementations. For example, the processor circuitry 44 may include any suitable device, such as a processor (e.g., CPU), programmable circuit, integrated circuit, memory and I/O circuits, an application specific integrated circuit, microcontroller, complex programmable logic device, other programmable circuits, or the like. The processor circuitry 44 may also include a non-transitory computer readable medium, such as random-access memory (RAM), a read-only memory (ROM), an erasable programmable read-only memory (EPROM or Flash memory), or any other suitable medium.

In one embodiment, the phototherapy device may include a sensor configured to sense biometric data of the skin. The processor circuitry 44 may receive an output of the sensor and control the properties of the phototherapeutic light 18 emitted by the light source 16 based on the output of the sensor. For example, the biometric sensor may be a temperature sensor for measuring a temperature of the skin. In this example, the processor circuitry 44 may reduce an output power of the light source 16 when the temperature exceeds a temperature threshold. Alternatively or additionally, the processor circuitry 44 may increase the output power of the light source 16 when the temperature is below the temperature threshold.

All ranges and ratio limits disclosed in the specification and claims may be combined in any manner. Unless specifically stated otherwise, references to “a,” “an,” and/or “the” may include one or more than one, and that reference to an item in the singular may also include the item in the plural.

Although the invention has been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A phototherapeutic footwear for treating vitamin D deficiency comprising:
a light source configured to emit phototherapeutic light including UVB light for stimulating vitamin D production and red light for stimulating melatonin production, wherein:
the UVB light includes light having a wavelength within a range of 300-315 nm; and
the red light includes light having a wavelength within a range of 620-700 nm;
a power source configured to store and supply electrical power to the light source;
a main body mechanically supporting the light source and the power source, wherein:
the main body includes a proximal end having an opening, a distal end, and an interior bounded by the main body and configured to receive a foot of a patient; and
the main body supports the light source such that the phototherapeutic light is emitted into the interior of the main body.

2. The phototherapeutic footwear of claim 1, wherein:
the light source includes a plurality of light emitters; and
the light emitters include UVB light emitters configured to emit the UVB light, and red light emitters configured to emit the red light.

3. The phototherapeutic footwear of claim 2, wherein the light emitters are spread across the main body such that the UVB light and the red light are received across different areas of the foot when inserted into the interior.

4. The phototherapeutic footwear of claim 3, wherein:
the main body is a slipper having a sole; and
the power source is housed within the sole.

5. The phototherapeutic footwear of claim 2, wherein:
the main body is a sock having a closed toe portion at the distal end opposite the opening;
the power source is located nearer the opening than the closed toe portion; and
the light emitters are spread across the main body portion and are electrically connected to the power source.

6. The phototherapeutic footwear of claim 2, further comprising processor circuitry configured to control the emission of the phototherapeutic light, wherein the processor circuitry controls the UVB light emitters separately from the red light emitters.

7. The phototherapeutic footwear of claim 2, wherein the light emitters further include blue light emitters configured to emit the blue light and a wavelength range of the blue light includes a wavelength of 405 nm.

8. The phototherapeutic footwear of claim 1 further comprising light guides extending from the light source towards at least one of the proximal end or the distal end, wherein:
the light guides are configured to receive the emitted phototherapeutic light from the light source, to transmit the phototherapeutic light via total internal reflection, and to emit the phototherapeutic light into the interior of the main body.

9. The phototherapeutic footwear of claim 8, wherein the light guides extend longitudinally along the main body portion.

10. The phototherapeutic footwear of claim 8, wherein the main body is made at least partially of a woven fabric and the light guides are interwoven into the woven fabric.

11. The phototherapeutic footwear of claim 8, wherein:
the main body is a sock having a closed toe portion at the distal end opposite the opening; and
the power source and the light source are located nearer the opening than the closed toe portion and the light guides extend from the light source towards the closed toe portion.

12. The phototherapeutic footwear of claim 8, wherein:
the main body is a slipper having a sole; and
the power source is housed within the sole.

13. The phototherapeutic footwear of claim 12, wherein the light guides extend circumferentially along the main body.

14. The phototherapeutic footwear of claim 1, further comprising processor circuitry configured to control the emission of the phototherapeutic light from the light source.

15. The phototherapeutic footwear of claim 14, wherein the processor circuitry is configured to separately control a dosage of the UVB light emitted by the light source and a dosage of the red light emitted by the light source.

16. The phototherapy footwear of claim 14, further comprising a sensor configured to detect when the foot is inserted into the opening, wherein:
the processor circuitry is further configured to stop the emission of the UVB light when the sensor detects that the foot is not inserted into the opening.

17. The phototherapeutic footwear of claim 16, wherein the processor circuitry is further configured to initiate emission of the phototherapeutic light when the sensor detects at a current time point that the foot is inserted into the opening after the sensor detected at a previous time point that the foot was not inserted into the opening.

18. The phototherapy footwear of claim 14, wherein the processor circuitry is further configured to:
measure a dosage of the UVB light emitted by the light source; and
when the measured dosage of the UVB light is greater than or equal to a UVB dosage threshold, stop the emission of the UVB light.

19. The phototherapy footwear of claim 14, wherein the processor circuitry is further configured to:
measure a dosage of the red light emitted by the light source; and
when the measured dosage of the red light is greater than or equal to a red dosage threshold, stop the emission of the red light.

20. The phototherapeutic light of claim 14, wherein the processor circuitry is further configured to:
determine a time of day;
compare the time of day to a red light initiation threshold; and
when the time of day is after the red light initiation threshold, cause the light source to emit the red light.
